(19) United States
(12) Patent Application Publication
(10) Pub. No.: US 2016/0016147 A1
(43) Pub. Date: Jan. 21, 2016

(54) ABSORBENT MEDIUM FOR ISOLATION OF BIOLOGICAL MOLECULES AND METHOD FOR SYNTHESIZING SAME

(71) Applicants: Zist Abzar Pajoohan, Tehran (IR); Fanavaran Nano-Meghyas, Tehran (IR)

(72) Inventors: Kamal Fatemi, Tehran (IR); Gholamreza Tavoosidana, Tehran (IR)

(21) Appl. No.: 14/868,371

(22) Filed: Sep. 28, 2015

Related U.S. Application Data
(60) Provisional application No. 62/057,211, filed on Sep. 29, 2014.

(51) Int. Cl. B01J 20/10; C12N 15/10; B01J 20/32; B01J 20/28; B01J 20/30; B01D 15/38; B01J 20/26

(52) U.S. Cl. CPC B01J 20/103; B01D 15/3819; C12N 15/101; B01J 20/261; B01J 20/28023; B01J 20/28028; B01J 20/3007; B01J 20/3295; B01J 20/3234; B01J 20/3214

(57) ABSTRACT

An absorbent medium for biological molecules separation is provided. The absorbent medium includes a scaffold made of polymeric nanofiber. The polymeric nanofiber is decorated with silica nanoparticles.

### CROSS REFERRENCE TO RELATED APPLICATION

The present application claims priority from pending U.S. Provisional Patent Application Ser. No. 62/057,211, filed Sep. 29, 2014, entitled “Nano-composite for isolating biological molecules”, the entire content which is incorporated herein by reference.

### SPONSORSHIP STATEMENT

This application has been sponsored by the Iranian Nanotechnology Initiative Council, which does not have any rights in this application.

### TECHNICAL FIELD

The present disclosure generally relates to an absorbent medium for isolation of biological molecules, a method for preparing the same, and the use thereof as a solid bed in isolation of biological molecules, such as nucleic acids.

### BACKGROUND

The isolation of DNA and RNA is an important step in biological and diagnostic processes. Thus, different methods for isolation and purification of nucleic acids from complex mixtures such as blood, serum, plasma, cerebrospinal fluid (CSF), tissue, etc., have been introduced. Generally, these methods include lysing of the biological materials by a detergent or a chaotropic salt, and possibly, in combination with enzymatic protein degradation, followed by a purification process.

After lysing the biological sample, a purification process is conducted with the aim of removing unwanted substances from the biological sample. Purification process of the biological samples can be carried out via solvent-based or solid-phase-based methods.

Conventional DNA isolation methods typically entail suspending cells in a solution and using enzymes and/or chemicals, to gently lyse the cells, thereby releasing the DNA contained within the cells into the resulting lysate solution. Many conventional protocols in use typically entail use of phenol or an organic solvent mixture containing phenol and chloroform to extract additional cellular material such as proteins and lipids from a conventional lys ate solution produced as described above. The phenol/chloroform extraction step is typically followed by precipitation of the nucleic acid material remaining in the extracted aqueous phase by adding ethanol to that aqueous phase. The precipitate is typically removed from the solution by centrifugation, and the resulting pellet of precipitate is allowed to dry before being resuspended in water or a buffer solution for further processing or analysis.

As is known to those skilled in the art, conventional nucleic acid isolation procedures have significant drawbacks. Among these drawbacks are the time required for the multiple processing steps necessary in the extractions and the dangers of using phenol or chloroform. Phenol causes severe bums on contact. Chloroform is highly volatile, toxic and flammable. Another undesirable characteristic of phenol/chloroform extractions is that the oxidation products of phenol can damage nucleic acids. Only freshly redistilled phenol can be used effectively, and nucleic acids cannot be left in the presence of phenol. Generally also, multi-step procedures are required to isolate RNA after phenol/chloroform extraction. In addition, ethanol (or isopropanol) precipitation must be employed to precipitate the DNA from a phenol/chloroform extracted aqueous solution of DNA, and remove residual phenol and chloroform from the DNA.

Therefore, a recognized need exists for methods that are simpler, safer, and more effective than the traditional phenol/chloroform extraction and ethanol precipitation methods to isolate DNA and/or RNA sufficiently for manipulation using molecular biology procedures.

Moreover, regarding the solid-phase-based extraction methods, different absorbent media have been introduced in the art. Silica particles and different silicon containing materials including boron silicate, aluminum silicate, phosphor silicate, silica carbonyl, silica sulfonyl and silica phosphonyl, have been used as the solid phase in the prior art. Moreover, a solid phase extraction method is disclosed in the art based on immobilizing the DNA onto diatomaceous earth in the presence of a chaotropic agent and eluting the DNA with water or low salt buffer, afterwards. The resulting purified DNA is biologically active, but separation of the absorbent solid phase from the media by centrifugation makes a rigid precipitate, hard to re-suspend again in multiple absorbing, washing and eluting steps.

Packed bed chromatography is commonly used in the bioseparation industry for capture of target proteins and other compounds. Packed bed chromatography is characterized by the use of resins, gels, beads or other particles that are packed in a column for capturing and eluting a liquid sample through such column The technique has, however, widely known drawbacks, such as, slow intra-particle diffusion, high pressure drops across the column bed, relatively slow throughput, and high cost of chromatography media. In addition, the costs of conventional bead-packed bed columns are typically high, which makes the development of disposable column systems a challenge.

There is a need for overcoming the drawbacks of the packed bed chromatography. More particularly, there are needs for increasing intra-particle diffusion and decreasing the pressure drop across the column There is also a need for increasing throughput during bioseparation. The cost of resins to perform large scale bioseparation is relatively high. There are further needs for reducing cost of bioseparation and for developing reusable or disposable bioseparation devices.

Packing of the solid phase in small spin-columns makes user friendly kits, but needs making the absorbent material in a filter format to be packed in columns As is known from the prior art, in order to increase the isolation yield, number of filters stacked in the spin column needs to be increased, which in turn, has the drawback of higher pressure drop and lower throughput. Many efforts have been made to formulate acceptable membranes to adsorb nucleic acids with high efficacy.

Modified glass fiber membranes have been introduced in the art, which exhibit sufficient hydrophilicity and sufficient electro positivity to bind DNA from a suspension containing DNA, and permit elution of the DNA from the membrane. Generally, the hydrophilic and electropositive characteristics are expressed at the surface of the modified glass fiber membrane. The membranes action in absorbing nucleic acids is acceptable, but the main drawback is the low extraction efficacy of this method.

Silane-treated silica filter media has been introduced in the art, prepared from rice hull ash or diatomaceous earth with functional quaternary ammonium group or functional sulphonate group. In this method, unwanted soluble materials are captured by the treated silica filter media, and desired components of interest are recovered from the flow-through. The extracted molecules are pure, but the problem is the complexity of the method.

Magnetically responsive particles or as they are simply called in the art, magnetic particles and methods for using magnetic particles have been developed for the isolation of nucleic acid materials. Several different types of magnetic particles designed for use in nucleic acid isolation are described in the prior art, and many of those types of particles are available from commercial sources. The most important advantage of using magnetic particles, which are typically used as magnetic beads, is that they can be used in fully automated procedures. The widely known drawback of using magnetic beads is the lower isolation yield compared to conventional spin column methods. Moreover, clogging or clamping the tip of the sampler is another known drawback while using magnetic beads. As is known to a person skilled in the art, when DNA or RNA are adsorbed on the magnetic particles, they cause the particles to agglomerate and clog the tip of the sampler.

There is a need recognized in the art for solid phase methods that are simpler, and more effective than the abovementioned methods to isolate DNA and/or RNA sufficiently for manipulation using molecular biology procedures.

Additionally, the industry is currently being driven by a need to replace the conventional downstream separation processes with disposable systems, which have the potential to decrease labor and operational expenses.

Hence, there is a need to provide a more efficient absorbent media, which can be used as a solid bed for the isolation of biological molecules, with a lower pressure drop through the bed and a higher throughput and isolation yield. There is also a need to introduce a solid bed capable of being used to form disposable column systems for use in fully automated systems.

### SUMMARY

The following brief summary is not intended to include all features and aspects of the present disclosure, nor does it imply that the disclosure must include all features and aspects discussed in this summary.

The present disclosure relates to an absorbent medium for isolation of biological molecules, a method for preparing the same, and the use thereof as a solid bed in isolation of biological molecules, such as nucleic acids. The absorbent medium for biological molecules separation presented in this disclosure includes a scaffold made of nanofiber (e.g., polymeric nanofiber) decorated with silica nanoparticles to improve the purification efficacy of different biomolecules including DNA and RNA from biological samples.

According to a preferred implementation of the present disclosure, the absorbent media, prepared pursuant to the teachings of the present disclosure is stacked in a separation column in the form of nonwoven membranes.

In one implementation of the present disclosure, silica nanoparticles with mesoporous structures, can be used to decorate the aforementioned polymeric nanofibers.

The polymeric nanofiber can be made of a polymer selected from the group consisting of polymethyl methacrylate (PMMA), polyvinyl alcohol (PVA), nylon, polystyrene (PS), polyamide, polyethylene terephthalate (PET), polybutylene terephthalate (PBT), polyethylene (PE), polypropylene (PP), polyolefin, polyethylene oxide (PEO), polyphenol formaldehyde (PPF), polyvinyl chloride (PVC), aromatic polyamide, polyacrylonitrile (PAN), polyurethane (PU), or combinations thereof. The silica nanoparticles may have mesoporous structure. The silica nanoparticles can be made using a silica source selected from the group consisting of tetraethyl orthosilicate (TEOS), 2-[methoxy(polyethyleneoxy)propyl]trimethoxysilane (PEGTMS), (3-glycidoxypropyl)trimethoxysilane (GPTES), triethoxysilane (APTES), trimethoxysilyl-propyl diethylene triamine (DETA), or combinations thereof. The nanofibers may have a diameter of less than 100 nanometer. The silica nanoparticles may have a diameter less than 100 nanometer. The absorbent medium may be in the form of a membrane. A solid bed for isolating a biological molecule is provided. The solid bed may include a plurality of the absorbent mediums stacked in a column

A method for synthesizing an absorbent medium including a polymeric nanofiber scaffold is provided. The method includes forming the polymeric nanofiber scaffold by electrospinning a polymeric solution. The method also includes electrospraying a silica source onto the polymeric nanofiber scaffold, when the electrospinning and the electrospraying are carried out simultaneously. The electrospraying of the silica source onto the polymeric nanofiber scaffold, may decorate the polymeric nanofiber scaffold with silica nanoparticles. The polymeric solution can be a PMMA solution with a preferred concentration of about 1 to about 5 percent by weight.

A method for isolating a biological molecule from a sample is provided. The method includes contacting the sample with an absorbent medium including a scaffold made of polymeric nanofiber decorated with silica nanoparticles. The method also includes allowing the biological molecule to bind to the absorbent medium and thereby be separated from the sample. The method further includes, upon allowing the biological molecule to bind to the absorbent medium and thereby be separated from the sample, retrieving the sample. The method also includes collecting the biological molecule bound to the absorbent medium by eluting through the absorbent medium an elution solution interfering with the binding between the biological molecule and the absorbent medium, so as to detach the biological molecule from the absorbent medium. The biological molecule can be a nucleic acid.

### BRIEF DESCRIPTION OF THE DRAWINGS

While the specification concludes with claims particularly pointing out and distinctly claiming the subject matter that is regarded as forming the present disclosure, it is believed that the disclosure will be better understood from the following description taken in conjunction with the accompanying drawings, where like reference numerals designate like structural and other elements, in which:

FIG. 1 illustrates a schematic diagram of an implementation of an apparatus used to form an absorbent medium, pursuant to the teachings of the present disclosure.

FIGS. 2A-2B illustrate scanning electron microscope (SEM) images of an exemplary absorbent medium, produced pursuant to the teachings of the present disclosure, with image resolutions of 5 μm (FIG. 2A) and 1 μm (FIG. 2B).

FIG. 3 illustrates an exemplary process of an exemplary solid bed in a spin column preparation using the absorbent medium, produced pursuant to the teachings of the present disclosure.

FIG. 4 illustrates gel electrophoresis of isolated genomic DNA from a sample of mouse liver, prepared pursuant to the teachings of the present disclosure.

FIG. 5 illustrates gel electrophoresis of a sample of RNA, isolated from a sample of mouse liver.

FIG. 6 illustrates optical density scan of DNA isolated by an exemplary absorbent medium, as described in more detail in connection with Example 1.

FIG. 7 illustrates optical density scan of RNA isolated by an exemplary absorbent medium, as described in more detail in connection with Example 1.

### DETAILED DESCRIPTION

The following detailed description is presented to enable any person skilled in the art to make and use the disclosure. For purposes of explanation, specific nomenclature is set forth to provide a thorough understanding of the present disclosure. However, it will be apparent to one skilled in the art that these specific details are not required to practice the teachings of the present disclosure.

Descriptions of specific applications are provided only as representative examples. Various modifications to the preferred implementations will be readily apparent to one skilled in the art, and the general principles defined herein may be applied to other implementations and applications without departing from the scope of the disclosure. The present disclosure is not intended to be limited to the implementations shown, but is to be accorded the widest possible scope consistent with the principles and features disclosed herein.

It should be understood by a person skilled in the art, that the disclosure described herein is directed to an absorbent medium for isolation of biological molecules, a method for preparing the same, and the use thereof as a solid bed in isolation of biological molecules, such as nucleic acids. It should, of course, be understood that aspects of the present disclosure may also be useful in related contexts, as is understood to those of skill in the pertinent arts.

The absorbent medium for biological molecules separation presented in this disclosure includes a scaffold made of nanofibers decorated with silica nanoparticles to improve the purification efficacy of different biomolecules including DNA and RNA from biological samples. It is noted that a scaffold made of nanofibers is essentially a polymeric nanofiber mat.

As is known to a person skilled in the art, fibers of small diameter or nanofibers have a high surface area to weight ratio. Therefore, using nanofibers as the scaffold in the absorbent medium, can increase the surface area to weight ratio of the absorbent medium.

As is known in the art, nanofibers are preferably utilized in separation devices in the form of a nonwoven material, most preferably in the form of one or more nonwoven membranes. According to a preferred implementation of the present disclosure, the absorbent media, prepared pursuant to the teachings of the present disclosure can be stacked in a column in the form of nonwoven membranes, as will be described in more detail in connection with Example 1.

Nanofibers can be prepared by different techniques, known in the art, such as, electrospinning, melt spinning, dry spinning, wet spinning, melt blowing, and extrusion methodologies.

In some implementations of the present disclosure, the aforementioned nanofibers used as the scaffold in the absorbent medium, can be produced using a variety of polymers, such as polymethyl methacrylate (PMMA), polyvinyl alcohol (PVA), nylon, polystyrene (PS), polyamide, polyethylene terephthalate (PET), polybutylene terephthalate (PBT), polyethylene (PE), polypropylene (PP), polyolefin, polyethylene oxide (PEO), polyphenol formaldehyde (PPF), polyvinyl chloride (PVC), aromatic polyamide, polyacrylonitrile (PAN), polyurethane (PU), or combinations thereof.

In one preferred implementation of the present disclosure, a PMMA solution with a preferred concentration of between about 1 and 5 percent by weight can be used as the electrospinning solution to prepare the aforementioned polymeric nanofibers.

The surface of the polymeric nanofibers, described, can be decorated by silica nanoparticles, which are electrosprayed onto the surface of the nanofibers, as will be described in more detail hereinbelow.

In preferred implementations of the present disclosure, different materials can be used as the silica source reagent in the electrospray process to produce silica nanoparticles, namely, tetraethyl orthosilicate (TEOS), 2-[methoxy(polyethyleneoxy)propyl]trimethoxysilane (PEGTMS), (3-glycidoxypropyl)trimethoxysilane (GPTES), triethoxysilane (APTES), trimethoxysilyl-propyl diethylenetriamine (DETA), or combinations thereof.

In one implementation of the present disclosure, silica nanoparticles with mesoporous structures, can be used to decorate the aforementioned polymeric nanofibers. Mesoporous silica nanoparticles are synthesized by the template polymerization of silicate around a surfactant mesophase.

Using surfactants in the silica source material can encourage self-assembly in the reagent electrosprayed on the polymeric nanofiber scaffold, and make mesoporous silica assemblies on the aforementioned polymeric nanofiber mat, but without surfactants the particles may grow without a mesoporous structure.

In a preferred implementation of the present disclosure, the aforementioned surfactant used to produce mesoporous silica nanoparticles, can be a surfactant such as cetyl trimethylammonium bromide (CTAB), or pluronic165.

As is known in the art, different types of surfactants can be used to achieve different arrays of silicates. The most common types of mesoporous silica nanoparticles, known in the art, are MCM-41, SBA-15, and MSU-type nanoparticles, which can be used in preferred implementations of the present disclosure, as the aforementioned mesoporous silica nanoparticles used to decorate the aforementioned polymeric nanofibers, described.

It should be understood, by a person skilled in the art that any known mesoporous structure of silica falls within the scope of the present disclosure.

According to preferred implementations of the present disclosure, the polymeric nanofiber scaffold of the present disclosure, can be prepared by the known technique of electrospinning, and the silica nanoparticles, which are used to decorate the polymeric nanofiber scaffold, can be formed by electrospraying the silica source onto the surface of the aforementioned polymeric nanofiber scaffold. The electrospinning and the electrospraying processes, mentioned, can be carried out simultaneously.

In preferred implementations of the present disclosure, the formation of the polymeric nanofiber scaffold by electrospinning is carried out simultaneously with the formation of silica nanoparticles by electrospraying, for example, while polymeric nanofibers are being formed by electrospinning, silica nanoparticles can be electrosprayed onto the surface of the nanofibers, layer by layer to ensure a complete decoration of the polymeric nanofibers with silica nanoparticles.

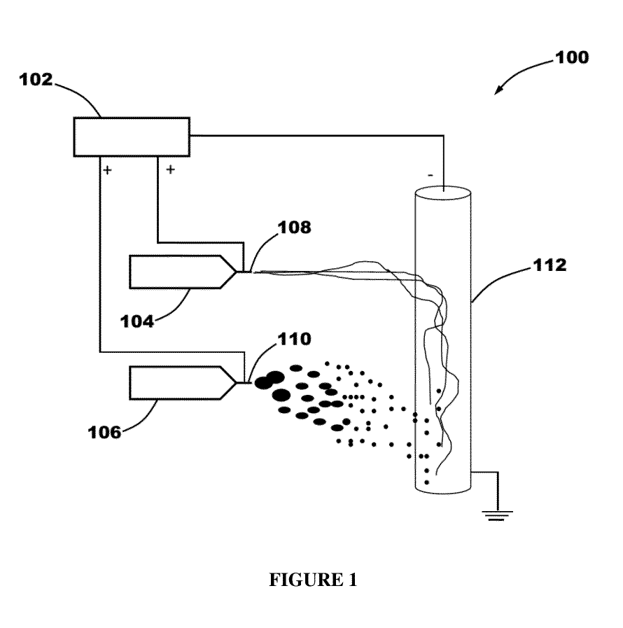

FIG. 1 of the drawings illustrates a schematic diagram of an implementation of the apparatus used to form the absorbent medium, pursuant to the teachings of the present disclosure. As is illustrated in FIG. 1, the electrospinning/electrospray apparatus 100 according to a preferred implementation of the present disclosure, includes two nozzles, designated by reference numerals 104 and 106, one nozzle 106 is used to spray the electrospray medium, which is the silica source, pursuant to the teachings of the present disclosure, and the other nozzle 104 is used for electrospinning. A high voltage source 102 is connected to the tip 108 of electrospinning nozzle 104 and the tip 110 of electrospraying nozzle 106, and a rotating collector 112. The rotating collector 112 can be placed preferably 1 to 30 cm away from the tip 108 of the electrospinning nozzle 104 and the tip 110 of electrospraying nozzle 106, as will be discussed in more detail in connection with EXAMPLE 1, hereinbelow. Typically, an electrical field strength, between 2 and 400 kV/m can be established by the high voltage source 102, which is also described in more detail in connection with EXAMPLE 1, hereinbelow.

A motion device can be used to move the nozzles 104 and 106, horizontally along the length of the rotating collector 112, in order to achieve a more uniform electrospun nanofiber scaffold, which is decorated with electrosprayed silica nanoparticles, pursuant to the teachings of the present disclosure.

The polymeric nanofibers decorated with silica nanoparticles, which are produced by the method described, can have different thicknesses.

The polymeric nanofibers decorated with silica nanoparticles, which are prepared pursuant to the teachings of the present disclosure, can be packed in a separation column as an absorbent media to capture nucleic acids from lysed samples, which can be then, washed and eluted with specific washing and eluting buffers and solvents to achieve purified nucleic acids.

Centrifugal force or vacuum could be used for transferring material through the absorbent media in the column, which is formed as described.

The absorbent media made by the process disclosed in the present disclosure, which is described, can be packed in plastic tips of typical samplers, used in the art, to suck the sample lysate and washing and elution buffers by the negative pressure of the aforementioned sampler and push them out by exerting a positive pressure.

The plastic tips packed with the aforementioned absorbent media, as described, can be used with hand held samplers to extract the nucleic acids manually or by automated liquid handling machines, which are typically used in the art.

Different samples such as bodily fluids such as blood, serum, plasma and cerebrospinal fluid (CSF) can be used to isolate nucleic acids by the absorbent media of the present disclosure. Samples from human, animal, plants and cultured media can be used.

In Example 1, preparation of the polymeric nanofiber scaffold decorated with silica nanoparticles is disclosed. In this implementation example, as will be discussed in more detail below, with further reference to FIG. 1 of the drawings, a polymer solution of 2.5 percent by weight polymethyl methacrylate (PMMA) in chloroform is jetted from nozzle 104, in a 10 kV/m electrical field onto the rotating collector 112, which is preferably placed 15 cm away from the tip 108 of nozzle 104. Another nozzle 106 containing the electrospray medium, which is the silica source reagent, simultaneously, jets or sprays the solution onto the forming nanofibers on the collector 112. The thickness of the absorbent media prepared by this method can be preferably controlled by the duration of the simultaneous electrospinning/electrospraying process, described.

The aforementioned silica source reagent can be prepared by mixing TEOS, ethanol, H2O, and HCl with molar ratios of (1:3:8:5×10-5), respectively. Then, the mixture can be refluxed for 1 hour followed by a 15 min stirring, and meanwhile, increasing the HCL concentration up to 7.34 molar and adding 1.5 to 5 percent mass/volume of CTAB to the mixture. The surfactant in the as-synthesized silica can be then removed via a chemical extraction, yielding a mesoporous material.

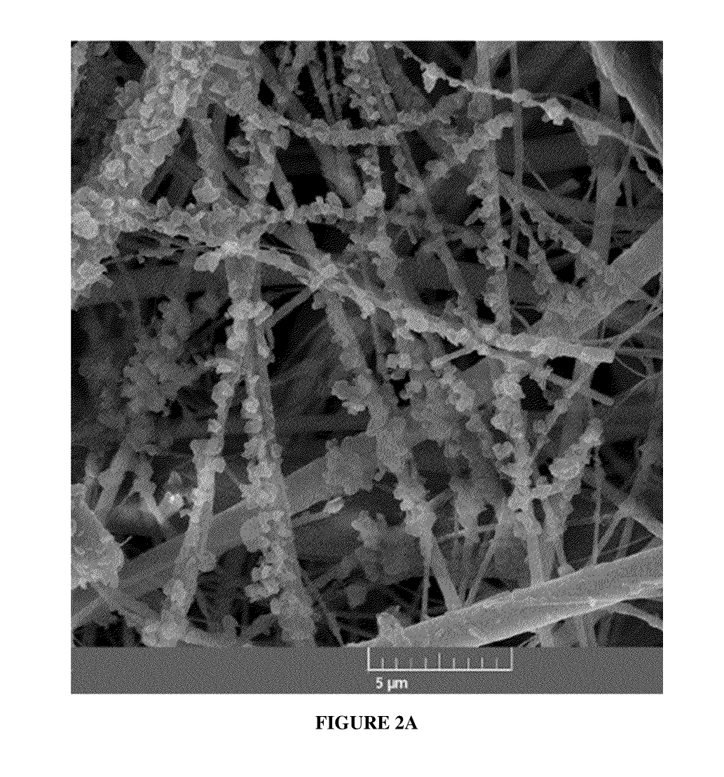

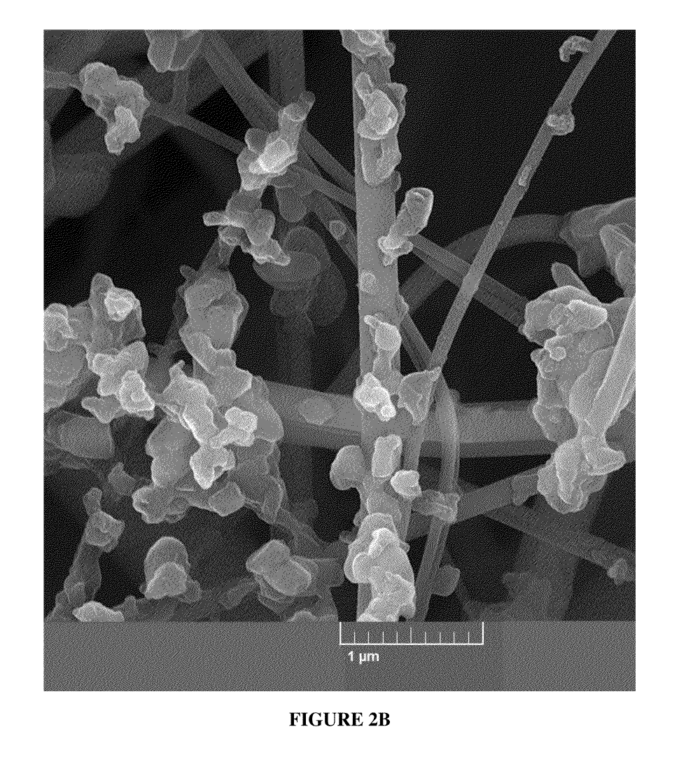

FIG. 2 of the drawings illustrates scanning electron microscope (SEM) images of exemplary absorbent medium produced pursuant to the teachings of the present disclosure, which confirms the uniform decoration of the polymeric nanofibers with silica nanoparticles.

The average diameters of the produced nanofibers and silica nanoparticles can be controllable via the conditions of the synthesis process. For example, the produced nanofibers and silica nanoparticles may have diameters less than 100 nanometer.

Two types of absorbent media can be made by the method described in Example 1, which differ in thickness. The first absorbent media, labeled as Nano-based 1 can be prepared by 15 minutes of the simultaneous electrospinning/electrospraying process described in Example 1, and the second absorbent media, labeled as Nano-based 2 can be prepared by the same method, as with Nano-based 1, but with a duration of 25 minutes of the simultaneous electrospinning/electrospraying process. Accordingly, Nano-based 2 is thicker than Nano-based 1. The image shown in FIG. 2A has an image resolutions of 5 μm and FIG. 2B shows the image with a 1 μm resolution.

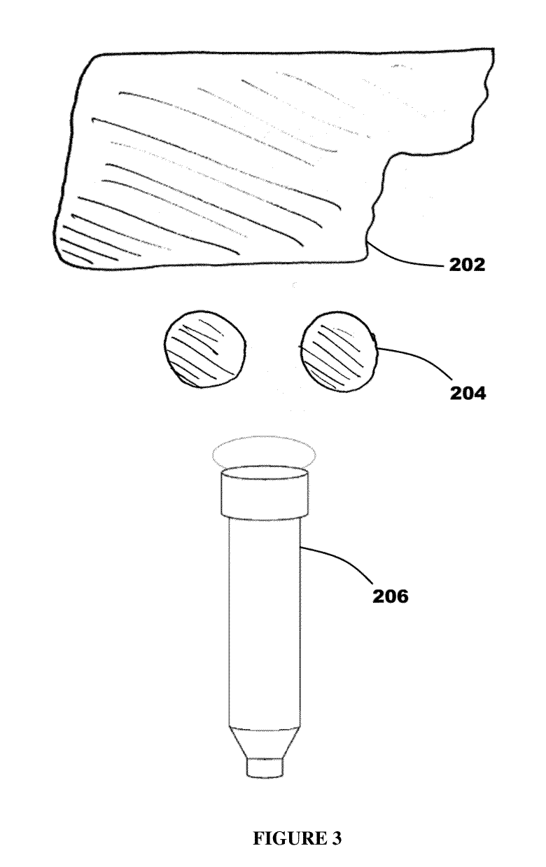

With reference to FIG. 3 of the drawings, the produced absorbent media 202, as described, can be formed into disks 204 by die cutting, and then, the disks 204 can be stacked in a spin column 206. Plastic rings can be used to fix the absorbent media disks in the spin column, as is the common practice in the art. Example 2 is an example of DNA and RNA isolation using the techniques disclosed herein.

In Example 2, the absorbent media, prepared as described in Example 1 is used for DNA and RNA isolation. A sample of mouse liver lysate was transferred into the spin column containing the absorbent media, and the desired target material was absorbed onto the absorbent media stacked inside the column. Then, as is a common practice in the pertinent art, the substances were washed to purify the target material using wash buffers, and finally, the target was eluted from the column.

Spectrophotometric data, as presented and set forth in TABLES 1 and 2, hereinbelow, are used to determine the efficiency of the DNA and RNA isolation, using the absorbent media, prepared by the method described in Example 1.

As is known to those skilled in the art, absorbance at a wavelength of 260 nanometer (nm) can be used to determine the concentration of a nucleic acid in a solution, while absorbance at a wavelength of 280 nanometer can be used to determine the concentration of protein in a solution. The ratio between the readings at a wavelength of 260 nanometer and a wavelength of 280 nanometer, can provide an estimate of the degree to which a given target nucleic acid has been isolated from proteins. As is known in the art, pure nucleic acid preparations have a 260/280 ratio of between 1.7 and 1.9. Moreover, the absorbance at a wavelength of 230 nanometer can be used to determine the concentration of chaotropic salts in a solution. The ratio between the readings at a wavelength of 260 nanometer and a wavelength of 230 nanometer, can provide an estimate of the degree to which a given target nucleic acid has been isolated from chaotropic salts. As is known in the art, pure nucleic acid preparations have a 260/230 ratio of between 1.7 and 1.9.

The data presented and set forth in TABLES 1 and 2, confirms the efficacy of the absorbent medium of the present disclosure, since the absorbance ratios of 260/280 and 260/230 for this absorbent medium, e.g., the purity of the nucleic acids isolated by the absorbent medium of the present disclosure is comparable to that of a Qiaamp DNA blood mini kit, hereinafter simply called “Qiagen standard kit”.

TABLE 1
SPECTROPHOTOMETRIC DATA FOR EXTERACTED DNA

| Absorbent medium | 260/280 ratio | 260/230 ratio |
|---|---|---|
| Standard (Qiagen Standard | 1.93 | 1.8 |
| Kit) | | |
| Nano based 1 | 1.83 | 1.91 |
| Nano based 2 | 1.94 | 1.84 |

TABLE 2
SPECTROPHOTOMETRIC DATA FOR EXTERACTED RNA

| Absorbent medium | 260/280 ratio | 260/230 ratio |
|---|---|---|
| Standard (Qiagen Standard | 1.77 | 1.97 |
| Kit) | | |
| Nano based 1 | 1.96 | 1.98 |
| Nano based 2 | 1.89 | 1.97 |

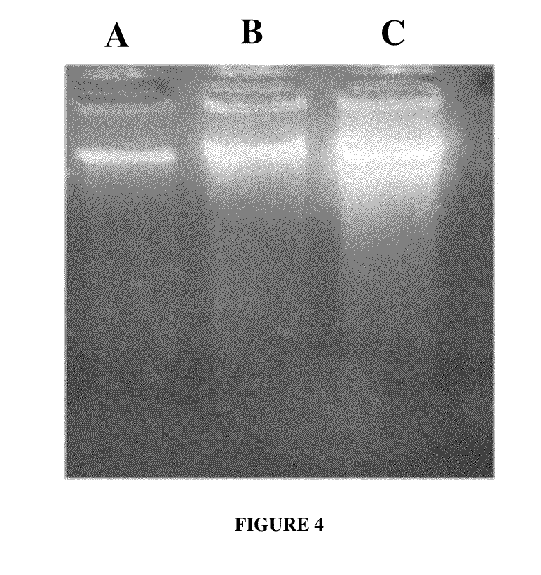

FIG. 4 of the drawings, illustrates gel electrophoresis of isolated genomic DNA from a sample of mouse liver. In FIG. 4, A illustrates genomic DNA extracted with a conventional silica micro-fiber filter; B illustrates genomic DNA extracted with a Qiagen standard kit; and C illustrates genomic DNA extracted with an exemplary absorbent medium, prepared pursuant to the teachings of the present disclosure. As can be seen in FIG. 4, the higher intensity of the bands in sample C, which was isolated using the absorbent medium of the present disclosure, shows the superior isolation capacity of the absorbent medium of the present disclosure compared to conventional standard kits for DNA isolation shown as A and B.

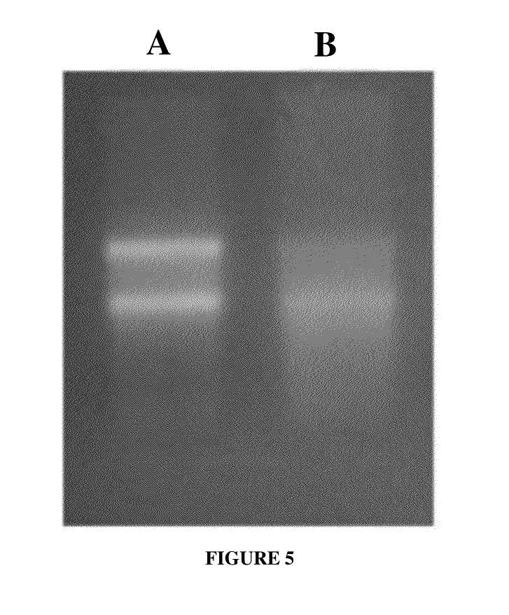

FIG. 5 of the drawings illustrates gel electrophoresis of a sample of RNA, isolated from a sample of mouse liver. In FIG. 5, A illustrates total RNA extracted with an exemplary absorbent medium, prepared pursuant to the teachings of the present disclosure; and B illustrates total RNA extracted with a Qiagen standard kit. Again, the higher intensity of the bands in sample A, which is isolated with the absorbent medium of the present disclosure, confirms the superior isolation capacity of the present disclosure compared to a Qiagen standard kit B.

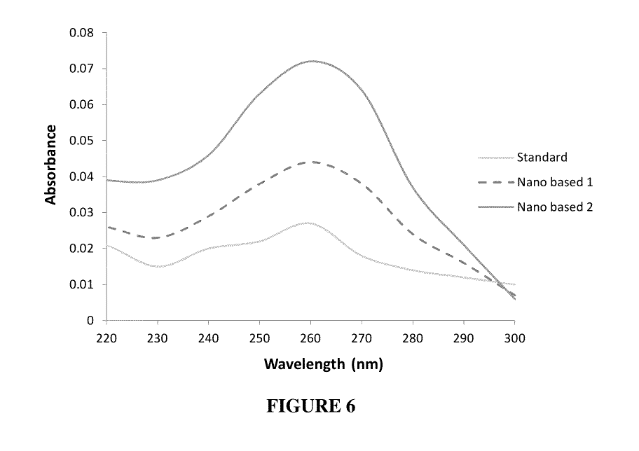

FIG. 6 of the drawings illustrates optical density scan of DNA isolated by the exemplary absorbent medium, as described in connection with Example 1. As can be seen in FIG. 6, the higher absorbance peak at 260 nanometer shows that a larger amount of DNA is isolated using the exemplary absorbent medium of the present disclosure. In addition, as shown in FIG. 6, the exemplary absorbent medium labeled as Nano-based 2, which is thicker than the exemplary absorbent medium labeled as Nano-based 1, has a better performance in the isolation of DNA.

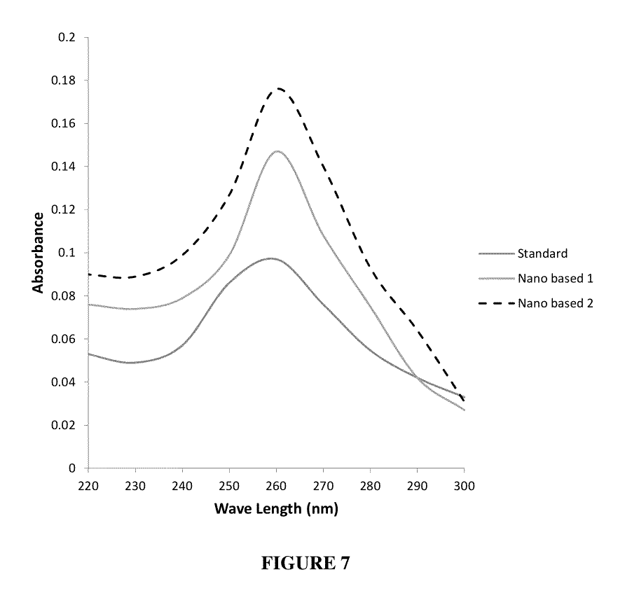

FIG. 7 of the drawings, similar to FIG. 6, illustrates optical density scan of RNA isolated by the exemplary absorbent medium, as described. The same results are obtained in this case, as was described in detail in connection with DNA isolation.

Due to the high efficacy of the absorbent medium of the present disclosure, a solid bed formed via stacking the absorbent medium of the present disclosure as nonwoven membranes in a spin column, as described in connection with Example 1 and illustrated in FIG. 3 of the drawings, can have a smaller volume, which eliminates the well-known drawback of the conventional spin columns, that is, the high pressure drop in the column.

While the present disclosure has been illustrated by the description of the implementations thereof, and while the implementations have been described in detail, it is not the intention of the applicant to restrict or in any way limit the scope of the appended claims to such detail. Additional advantages and modifications will readily appear to those skilled in the art. Therefore, the disclosure in its broader aspects is not limited to the specific details, representative apparatus and method, and illustrative examples shown and described. Accordingly, departures may be made from such details without departure from the breadth or scope of the applicant's concept. Furthermore, although the present disclosure has been described in connection with a number of exemplary implementations and implementations, the present disclosure is not so limited but rather covers various modifications and equivalent arrangements, which fall within the purview of the appended claims.

What is claimed is:

1. An absorbent medium comprising a scaffold made of polymeric nanofiber, wherein, the polymeric nanofiber is decorated with silica nanoparticles.

2. The absorbent medium of claim 1, wherein the polymeric nanofiber is made of a polymer selected from a group consisting of polymethyl methacrylate (PMMA), polyvinyl alcohol (PVA), nylon, polystyrene (PS), polyamide, polyethylene terephthalate (PET), polybutylene terephthalate (PBT), polyethylene (PE), polypropylene (PP), polyolefin, polyethylene oxide (PEO), polyphenol formaldehyde (PPF), polyvinyl chloride (PVC), aromatic polyamide, polyacrylonitrile (PAN), polyurethane (PU), or combinations thereof.

3. The absorbent medium of claim 1, wherein the silica nanoparticles have a mesoporous structure.

4. The absorbent medium of claim 1, wherein the silica nanoparticles are made using a silica source selected from a group consisting of tetraethyl orthosilicate (TEOS), 2-[methoxy(polyethyleneoxy)propyl]trimethoxysilane (PEGTMS), (3-glycidoxypropyl)trimethoxysilane (GPTES), triethoxysilane (APTES), trimethoxysilyl-propyl diethylene triamine (DETA), or combinations thereof.

5. The absorbent medium of claim 1, wherein the polymeric nanofiber has a diameter of less than 100 nanometer.

6. The absorbent medium of claim 1, wherein the silica nanoparticles have a diameter less than 100 nanometer.

7. The absorbent medium of claim 1, wherein the absorbent medium is in the form of a membrane.

8. A solid bed for isolating a biological molecule, the solid bed comprising a plurality of the absorbent mediums according to claim 7, stacked in a column.

9. A method for synthesizing an absorbent medium including a polymeric nanofiber scaffold, the method comprising:
forming the polymeric nanofiber scaffold by electrospinning a polymeric solution; and
electrospraying a silica source onto the polymeric nanofiber scaffold, wherein, the electrospinning and the electrospraying are carried out simultaneously.

10. The method of claim 9, wherein the polymeric nanofiber scaffold is made of a polymer selected from a group consisting of polymethyl methacrylate (PMMA), polyvinyl alcohol (PVA), nylon, polystyrene (PS), polyamide, polyethylene terephthalate (PET), polybutylene terephthalate (PBT), polyethylene (PE), polypropylene (PP), polyolefin, polyethylene oxide (PEO), polyphenol formaldehyde (PPF), polyvinyl chloride (PVC), aromatic polyamide, polyacrylonitrile (PAN), polyurethane (PU), or combinations thereof.

11. The method of claim 9, wherein the silica source is selected from a group consisting of tetraethyl orthosilicate (TEOS), 2-[methoxy(polyethyleneoxy)propyl]trimethoxysilane (PEGTMS), (3-glycidoxypropyl)trimethoxysilane (GPTES), triethoxysilane (APTES), trimethoxysilyl-propyldiethylene triamine (DETA), or combinations thereof.

12. The method of claim 9, wherein electrospraying of the silica source onto the polymeric nanofiber scaffold decorates the polymeric nanofiber scaffold with silica nanoparticles.

13. The method of claim 12, wherein the silica nanoparticles have a mesoporous structure.

14. The method of claim 12, wherein the silica nanoparticles have a diameter of less than 100 nanometer.

15. The method of claim 9, wherein nanofibers included in the polymeric nanofiber scaffold have a diameter of less than 100 nanometer.

16. The method of claim 9, wherein the polymeric solution is a PMMA solution with a preferred concentration of about 1 to about 5 percent by weight.

17. A method for isolating a biological molecule from a sample, comprising:
contacting the sample with an absorbent medium including a scaffold made of polymeric nanofiber decorated with silica nanoparticles; and
allowing the biological molecule to bind to the absorbent medium and thereby be separated from the sample.

18. The method of claim 17, further comprising:
retrieving the sample after the biological molecule is bound to the absorbent medium and thereby separated from the sample; and
collecting the biological molecule bound to the absorbent medium by eluting through the absorbent medium an elution solution interfering with binding between the biological molecule and the absorbent medium, so as to detach the biological molecule from the absorbent medium.

19. The method of claim 17, wherein the biological molecule is a nucleic acid.

20. The method of claim 17, wherein the absorbent medium is synthesized via a method, comprising:
forming the scaffold by electrospinning a polymeric solution; and
electrospraying a silica source onto the scaffold,
wherein the electrospinning and the electrospraying are carried out simultaneously.

21. The method of claim 17, wherein the scaffold is made of a polymer selected from a group consisting of polymethyl methacrylate (PMMA), polyvinyl alcohol (PVA), nylon, polystyrene (PS), polyamide, polyethylene terephthalate (PET), polybutylene terephthalate (PBT), polyethylene (PE), polypropylene (PP), polyolefin, polyethylene oxide (PEO), polyphenol formaldehyde (PPF), polyvinyl chloride (PVC), aromatic polyamide, polyacrylonitrile (PAN), polyurethane (PU), or combinations thereof.

22. The method of claim 17, wherein the silica nanoparticles are made using a silica source selected from a group consisting of tetraethyl orthosilicate (hereinafter “TEOS”), 2-[methoxy(polyethyleneoxy)propyl]trimethoxysilane (PEGTMS), (3-glycidoxypropyl)trimethoxysilane (GPTES), triethoxysilane (APTES), trimethoxysilyl-propyldiethylene triamine (DETA), or combinations thereof.

23. The method of claim 17, wherein the silica nanoparticles have a mesoporous structures.

24. The method of claim 17, wherein the silica nanoparticles have a diameter of less than 100 nanometer.

25. The method of claim 17, wherein the nanofibers included in the scaffold have a diameter of less than 100 nanometer.

26. The method of claim 17, wherein the absorbent medium is a PMMA solution with a concentration of about 1 to about 5 percent by weight.
